(19) United States
(12) Patent Application Publication
(10) Pub. No.: US 2008/0039820 A1
(43) Pub. Date: Feb. 14, 2008

(54) Medical Device With Septum

(76) Inventors: Jeff Sommers, San Antonio, TX (US); Andrew D. Jacobson, San Antonio, TX (US)

Correspondence Address: SKOKOS LAW GROUP, LLC, 1100 JORIE BOULEVARD, SUITE 220, OAK BROOK, IL, 60523, US

(21) Appl. No.: 11/836,738

(22) Filed: Aug. 9, 2007

Related U.S. Application Data
(60) Provisional application No. 60/836,771, filed on Aug. 10, 2006.
(60) Provisional application No. 60/878,269, filed on Jan. 2, 2007.

(51) Int. Cl. A61M 39/02

(52) U.S. Cl. 604/539

(57) ABSTRACT

An improved septum-containing device having at least one polymeric septum with at least one corresponding plate having at least one aperture through which a septum-piercing device, usually a non-coring needle, may pass. The aperture exerts force on the non-coring needle to reduce the occurrence of inadvertent needle dislodgement from a septum-containing device. In a preferred embodiment the aperture is sized such that when the indwelling needle no longer is essentially perpendicular to the aperture, the side wall structure of the aperture exerts frictional forces on the needle. The present invention may be directed towards an implanted septum-containing device such as an infusion port as well as a non-implanted, externalized device with a polymeric septum such as a luer injection cap.

### CROSS REFERENCE TO RELATED APPLICATION

The present application is a regular utility application claiming the benefits of the filing date of provisional application, Application No. 60/836,771, filed on Aug. 10, 2006, by the present applicant and provisional application, Application No. 60/878,269, filed on Jan. 2, 2007, by the present applicant.

### BACKGROUND OF THE INVENTION

The present invention relates generally to a medical device containing a polymeric septum and more specifically such a device with a cover, plate or insert with at least one aperture which reduces the risk of premature dislodgement of a septum-piercing device from a septum-containing device.

The present invention is directed to septum-containing device used in human and veterinary medicine and research. Human and veterinary medical personnel use a variety of septum-containing devices which are both implantable in the body and externalized outside the body. The most common externalized septum-containing device in this setting is a rubber injection port or injection site placed in-line with various devices used to manage fluids passing in and out of a human or animal. Implanted septum-containing devices include, for example, implanted infusion ports and implanted infusion pumps.

Septum-containing devices are accessed with a septum-piercing device which usually is a non-coring needle. The non-coring needle is used to pierce the polymeric septum of the septum-containing device. In certain septum-containing device applications, it is desirable that the non-coring needle be left indwelling in the septum-containing device for an extended period of time (hours or days); that is, not used for a quick bolus infusion into the septum-containing device. A significant complication of septum-containing device use is premature dislodgement of an indwelling septum-piercing device. The present invention is designed to reduce the risk of premature dislodgement of a septum-piercing device in both externalized and implanted septum-containing devices.

Background art comprising implanted polymeric septum-containing device and systems addressing the problem of premature dislodgement are disclosed in U.S. Pat. Nos. 5,637,088; 4,861,341; 4,784,646; 4,781,695; 4,464,178. Implanted devices comprising an aperture precluding the use of a polymeric septum are disclosed in U.S. Pat. Nos. 6,544,214; 6,007,516; 5,989,239. U.S. Pat. No. 6,039,712 describes a polymeric septum-containing device with a wire mesh reinforcement disc. The above patents do not disclose use of a non-implanted (external) device.

### SUMMARY OF THE INVENTION

The present invention is directed to an improved septum-containing device having at least one polymeric septum with at least one corresponding cover, plate or insert, with at least one aperture through which a septum-piercing device may pass. The present invention may be directed towards an implanted septum-containing device such as an implanted infusion port as well as an externalized device with a septum such as an injection cap.

In one embodiment of the invention there may be a single cover, plate or insert which may be located either on the exterior surface of the septum, on the interior surface of the septum, or within the body of the septum. In another embodiment of the invention, multiple covers, plates or inserts may be located on the exterior surface, interior surface and/or within the body of the septum. The at least one aperture, may be round, square, or hexagonal in shape, or may be any other conventional shape, or may be a non-conventional shape. The aperture may have a featureless wall structure or may include threads or other features.

In a preferred embodiment, the side wall structure of the aperture in the invention exerts force on, for example, a non-coring needle to reduce the risk of premature dislodgement from a septum-containing device. A person may pass a non-coring needle through an aperture such that the non-coring needle is essentially perpendicular to the aperture. During prolonged dwelling in the septum-containing device, the non-coring needle may begin to shift, move, or rotate away from a position of being essentially perpendicular to the aperture which may lead to premature dislodgement of the non-coring needle. In the present invention, the aperture in the plate is sized such that when the indwelling non-coring needle begins to shift, move, or rotate away from a position of being essentially perpendicular to the aperture, the side wall structure of the aperture will exert frictional forces on the non-coring needle to oppose the movement of the non-coring needle thereby reducing its tendency to prematurely dislodge.

Alternatively, the side wall structure of the aperture may have means for locking the non-coring needle into the aperture including but not limited to threads which correspond to threads on a non-coring needle. Such means for locking further reduce the occurrence of premature dislodgement.

### BRIEF DESCRIPTION OF THE DRAWINGS

FIG. 1a is top view of an improved septum-containing device designed in accordance with an embodiment of the present invention, the figure illustrating an improved septum-containing device having one polymeric septum with one corresponding plate located on the exterior surface of the septum and one aperture through which a septum-piercing device may pass.

FIG. 1b is perspective view of an improved septum-containing device designed in accordance with an embodiment of the present invention, the figure illustrating an improved septum-containing device having one polymeric septum with one corresponding plate located on the exterior surface of the septum and one aperture through which a septum-piercing device has passed.

FIG. 1c is side cross sectional view of an improved septum-containing device designed in accordance with an embodiment of the present invention, the figure illustrating an improved septum-containing device having one polymeric septum with one corresponding plate located on the exterior surface of the septum and one aperture through which the non-coring needle or septum-piercing device has passed.

FIG. 2 is a side cross sectional view of an improved septum-containing device designed in accordance with an embodiment of the present invention, the figure illustrating an improved septum-containing device having one polymeric septum with one corresponding plate located on the exterior surface of the septum wherein the plate has a plurality of apertures where the non-coring needle or septum-piercing device has passed through one such aperture.

FIG. 3 is a side cross sectional view of an improved septum-containing device designed in accordance with an embodiment of the present invention, the figure illustrating an improved septum-containing device having one polymeric septum with two corresponding plates located within the body of the septum wherein the plates have a plurality of apertures where the non-coring needle or septum-piercing device has passed through one such aperture.

FIG. 4 is a side cross sectional view of an improved septum-containing device designed in accordance with an embodiment of the present invention, the figure illustrating an improved septum-containing device having one polymeric septum with one corresponding plate located on the interior surface of the septum.

FIG. 5 is a side cross sectional view of an improved septum-containing device designed in accordance with an embodiment of the present invention, the figure illustrating an improved septum-containing device having one polymeric septum with two corresponding plates, one plate located within the body of the septum and the other on the exterior surface of the septum wherein the plates have a plurality of apertures where the non-coring needle or septum-piercing device has passed through one such aperture.

FIG. 6a is a perspective view of a perforated plate of the present invention having a plurality of circular shaped apertures.

FIG. 6b is a top view of a perforated plate of the present invention having a plurality of circular shaped apertures.

FIG. 6c is a cut away view of a perforated plate of the present invention having a plurality of circular shaped apertures.

FIG. 6d is a top view of a perforated plate of the present invention having a plurality of hexagonal shaped apertures.

FIG. 6e is a cut away view of a perforated plate of the present invention having a plurality of hexagonal shaped apertures.

FIG. 6f is a top view of a perforated plate of the present invention having a plurality of rectilinear shaped apertures.

FIG. 6g is a cut away view of a perforated plate of the present invention having a plurality of rectilinear shaped apertures.

FIG. 6h is a top view of a plate of the present invention having a single aperture.

FIG. 6i is a cut away view of a plate of the present invention having a single circular aperture with a chamfered lead-in.

FIG. 7a is a top view of a component of an improved septum-containing device designed in accordance with an embodiment of the present invention, the figure illustrating an improved septum-containing device, namely an implanted infusion port wherein the top of the implanted infusion port has a plurality of apertures. The plate in this embodiment is integrated into the top of the implanted infusion port.

FIG. 7b is a perspective view of a perforated plate integrated implanted infusion port component of the present invention.

FIG. 7c is a cut away view of a perforated plate integrated implanted infusion port component of the present invention.

FIG. 8a is a top view of a slotted plate of the present invention.

FIG. 8b is a top view of two slotted plates of the present invention, wherein a first slotted plate has been placed over a second slotted plate wherein the first plate has been rotated 90 degrees relative to the second slotted plate.

FIG. 8c is a top view of a slotted plate of the present invention.

FIG. 9 is a side cut away view of a perforated plate of the present invention wherein a non-coring needle or septum-piercing device has been introduced through one aperture and is secured within the aperture through an interference fit.

FIG. 10a is a top view of an improved septum-containing device designed in accordance with another embodiment of the present invention, the figure illustrating an improved septum-containing device having one polymeric septum with one corresponding plate located on the exterior surface of the septum and one aperture that is threaded and matches to a corresponding thread on the exterior surface of the non-coring needle shaft to secure the non-coring needle in the threaded aperture.

FIG. 10b is a perspective view of an improved septum-containing device designed in accordance with another embodiment of the present invention, the figure illustrating an improved septum-containing device having one polymeric septum with one corresponding plate located on the exterior surface of the septum and one aperture that is threaded and matches to a corresponding thread on the exterior surface of the non-coring needle shaft to secure the non-coring needle in the threaded aperture.

FIG. 10c is a side cut away view of an improved septum-containing device designed in accordance with another embodiment of the present invention, the figure illustrating an improved septum-containing device having one polymeric septum with one corresponding plate located on the exterior surface of the septum and one aperture that is threaded and matches to a corresponding thread on the exterior surface of the non-coring needle shaft to secure the non-coring needle in the threaded aperture.

FIG. 11a is a top view of an improved septum-containing device designed in accordance with another embodiment of the present invention, the figure illustrating an improved septum-containing device having one polymeric septum with two covers, plates or inserts incorporated which include one top plate which has a single aperture and a second plate which is a “split” plate with spring-like properties.

FIG. 11b is a side cut away view of an improved septum-containing device designed in accordance with another embodiment of the present invention, the figure illustrating an improved septum-containing device having one polymeric septum with two covers, plates or inserts incorporated which include one top plate which has a single aperture and a second plate which is a “split” plate with spring-like properties.

FIG. 11c is a perspective view of an improved septum-containing device designed in accordance with another embodiment of the present invention, the figure illustrating an improved septum-containing device having one polymeric septum with two covers, plates or inserts incorporated which include one top plate which has a single aperture and a second plate which is a “split” plate with spring-like properties.

FIG. 12a is a top view of an improved septum-containing device designed in accordance with another embodiment of the present invention, the figure illustrating an improved septum-containing device having one polymeric septum with a cover, plate or insert having a magnet located under the surface of the cover, plate or insert which attracts a magnet attached to the shaft of a non-coring needle in order to create a coupling to lock or secure the non-coring needle in the septum-containing device.

FIG. 12b is a side cut away view of an improved septum-containing device designed in accordance with another embodiment of the present invention, the figure illustrating an improved septum-containing device having one polymeric septum with a cover, plate or insert having a magnet located under the surface of the cover, plate or insert which attracts a magnet attached to the shaft of a septum-piercing device in order to create a coupling to lock or secure the septum-piercing device in the septum-containing device.

FIG. 12c is a perspective view of an improved septum-containing device designed in accordance with another embodiment of the present invention, the figure illustrating an improved septum-containing device having one polymeric septum with a cover, plate or insert having a magnet located under the surface of the cover, plate or insert which attracts a magnet attached to the shaft of a non-coring needle in order to create a coupling to lock or secure the non-coring needle in the septum-containing device.

FIG. 13a is a top view of an improved septum-containing device designed in accordance with another embodiment of the present invention, the figure illustrating an improved septum-containing device having one polymeric septum with a cover, plate or insert located on the exterior surface of the septum and a reservoir with an angled section which causes an inserted non-coring needle to deform as it is advancing into the reservoir from its original orientation perpendicular to the cover, plate or insert.

FIG. 13b is a side cut away view of an improved septum-containing device designed in accordance with another embodiment of the present invention, the figure illustrating an improved septum-containing device having one polymeric septum with a cover, plate or insert located on the exterior surface of the septum and a reservoir with an angled section which causes an inserted non-coring needle to deform as it is advancing into the reservoir from its original orientation perpendicular to the cover, plate or insert.

FIG. 13c is a perspective view of an improved septum-containing device designed in accordance with another embodiment of the present invention, the figure illustrating an improved septum-containing device having one polymeric septum with a cover, plate or insert located on the exterior surface of the septum and a reservoir with an angled section which causes an inserted non-coring needle to deform as it is advancing into the reservoir from its original orientation perpendicular to the cover, plate or insert.

FIG. 14a is a top view of an improved septum-containing device designed in accordance with another embodiment of the present invention, the figure illustrating an improved septum-containing device having a cover, plate or insert with a single aperture located on the exterior surface of the septum which guides the non-coring needle to an electrical connector in the reservoir of the septum-containing device to create an electrical connection between the non-coring needle and a conductive lead extending from the septum-containing device.

FIG. 14b is a side cut away view of an improved septum-containing device designed in accordance with another embodiment of the present invention, the figure illustrating an improved septum-containing device having a cover, plate or insert with a single aperture located on the exterior surface of the septum which guides the non-coring needle to an electrical connector in the reservoir of the septum-containing device to create an electrical connection between the non-coring needle and a conductive lead extending from the septum-containing device.

FIG. 14c is a perspective view of an improved septum-containing device designed in accordance with another embodiment of the present invention, the figure illustrating an improved septum-containing device having a cover, plate or insert with a single aperture located on the exterior surface of the septum which guides the non-coring needle to an electrical connector in the reservoir of the septum-containing device to create an electrical connection between the non-coring needle and a conductive lead extending from the septum-containing device.

FIG. 15 is a cross sectional view of an improved septum-containing device designed in accordance with another embodiment of the present invention, the figure illustrating an improved septum-containing device having a cover, plate or insert with a penetrable cover located on the exterior surface of the cover, plate or insert.

FIG. 16a is a cross sectional view of an improved septum-containing device showing an indwelling septum-piercing device in an essentially perpendicular position relative to the aperture through which it passes. This figure illustrates the clearance of the septum-piercing device in the aperture.

FIG. 16b is a cross sectional view of an improved septum-containing device showing an indwelling septum-piercing device in an essentially non-perpendicular position relative to the aperture through which it passes. This figure illustrates the exertion of forces by the top and bottom of the side wall structure on the non-coring needle.

### DETAILED DESCRIPTION OF THE DRAWINGS

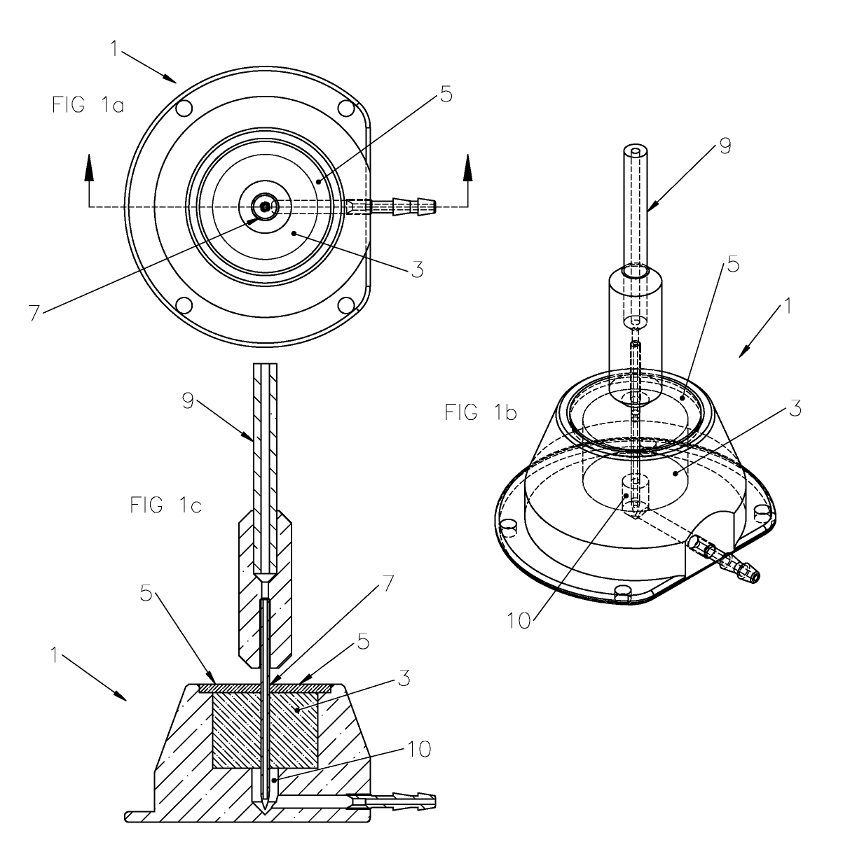

FIGS. 1a,b,c and 2 illustrate one embodiment of the present invention of a improved septum-containing device, shown generally at 1, having at least one polymeric septum 3 with at least one corresponding cover, plate or insert 5, with at least one aperture 7 through which a septum-piercing device 9, such as a non-coring needle, may pass. As shown in FIGS. 1a,b,c and 2, the at least one cover, plate or insert 5, may be located on the exterior surface of the septum 3, the surface of the septum 3 located most distal to the reservoir 10, with the at least one aperture 7. The presence of the at least one cover, plate or insert 5, with at least one aperture 7 in the present invention reduces the risk of premature dislodgement in an externalized and implanted septum-containing device.

In the present invention, the at least one polymeric septum 3 may be solid as commonly used in conventional septum-containing devices such as implanted infusion ports. Alternatively, the at least one septum 3 may have at least one orifice, aperture or slit. Alternatively, the at least one septum 3 may comprise two or more stacked layers. The at least one polymeric septum 3 and the reservoir 10 may be oriented along the same plane as the skin 2, oriented perpendicular to the skin 2, or oriented at angle between the same plane and perpendicular to skin 2. The septum-containing device 1 may comprise one reservoir 10 with at least one polymeric septum 3. Alternatively the septum-containing device 3 may comprise multiple reservoirs 10 each with at least one polymeric septum 3.

The septum-piercing device 9 maybe a non-coring, deflected tip (Huber) needle. Alternatively, the septum-piercing device 9 may be a non-coring pencil point needle with a venting fenestration near the pencil point. Alternatively, the septum-piercing device 9 may be a non-coring assembly comprising an obturating pencil point stylet inserted into a coring needle. Alternatively, the septum-piercing device 9 may be a non-coring assembly comprising an obturating pencil point stylet inserted into a polymeric cannula.

One significant cause of premature dislodgement is a result of the lateral (relative to the skin) forces acting on a septum-piercing device. Such lateral forces include movement of the skin and tissue overlying the septum-containing device, lateral forces acting on tubing attached to a septum-piercing device, and dressings or restraining devices (such as a lab animal jacket or harness) overlying the septum-piercing device especially when an implanted septum-containing device is sutured to, anchored to, or encapsulated in tissue. In certain septum-containing device applications, after a non-coring needle is used to pierce or puncture the septum of a septum-containing device, it is desirable that the non-coring needle be left indwelling in the septum-containing device for an extended period of time (hours or days). In such applications, the indwelling non-coring needle should ideally remain largely perpendicular to the septum-containing device. The occurrence of lateral forces, however, causes the indwelling non-coring needle to list. List(ing) can be defined as a lean, tilt, or otherwise significant deviation of the non-coring needle from its substantially perpendicular orientation relative to the septum-containing device, such as with a nautical ship that may list or tilt to one side. As the non-coring needle changes its angle away from perpendicularity relative to the septum-containing device, the lateral forces begin to align with the axis of the non-coring needle causing dislodgement forces (forces parallel to the line of action of the non-coring needle) to be exerted on the non-coring needle. Repetitive, multidirectional lateral forces can cause the eventual premature dislodgement of the non-coring needle. The presence of the at least one cover, plate or insert 5, with at least one aperture, opening, slot or through-hole through 7 in the present invention prevents the non-coring needle from listing excessively, thus countering a mechanism of premature dislodgement.

In the present invention, a septum-containing device, as shown in FIGS. 1a,b,c, specifically an implanted infusion port, designed with a cover, plate or insert 5 having a single aperture 7 allows for the use of a smaller reservoir 10 under the septum 3 compared to the reservoir of a conventional implanted infusion port. The diameter of the at least one aperture 7 (or length and width of a slot or non-circular aperture) of the cover, plate or insert 5 determines the area of the reservoir 9 of the improved septum-containing device. After the septum-piercing device or non-coring needle has punctured the septum 3, the reservoir 10 is where the non-coring needle tip resides so only a small reservoir is needed to accommodate the non-coring needle tip. By comparison, a conventional implanted infusion port typically has a large septum which allows for the non-coring needle to be inserted over a much larger septum area thus requiring a larger reservoir to accommodate the larger area where the non-coring needle tip may reside under the septum. A smaller reservoir may be preferred because it allows for more efficient flushing and evacuation of the reservoir as less fluid is needed to flush and evacuate the fluid contents of the reservoir.

A surprising discovery from the development and testing of the present invention revealed that repeated piercing and removal in and out of an implanted infusion port septum comprising a plate, cover or insert 5 having multiple apertures 7 by a 22 gauge non-coring pencil point needle 9 with venting fenestration resulted in a septum 3 which had less visible damage (i.e., deformed septum surfaces, shards of polymer) than that seen on a polymeric septum of a similarly sized conventional implanted infusion port using the same style needle. This result is surmised to be due to the fact that the aperture 7 forces the non-coring needle 9 to more perpendicularly pierce the septum 3 while a conventional implanted infusion port accessed by such a needle is not similarly constrained to such perpendicular piercing. In the conventional implanted infusion port, the less perpendicular piercing yielded intersecting piercing tracks through the septum which also led to leaking of the conventional implanted infusion port after far fewer piercings than in an implanted infusion port built according to the invention.

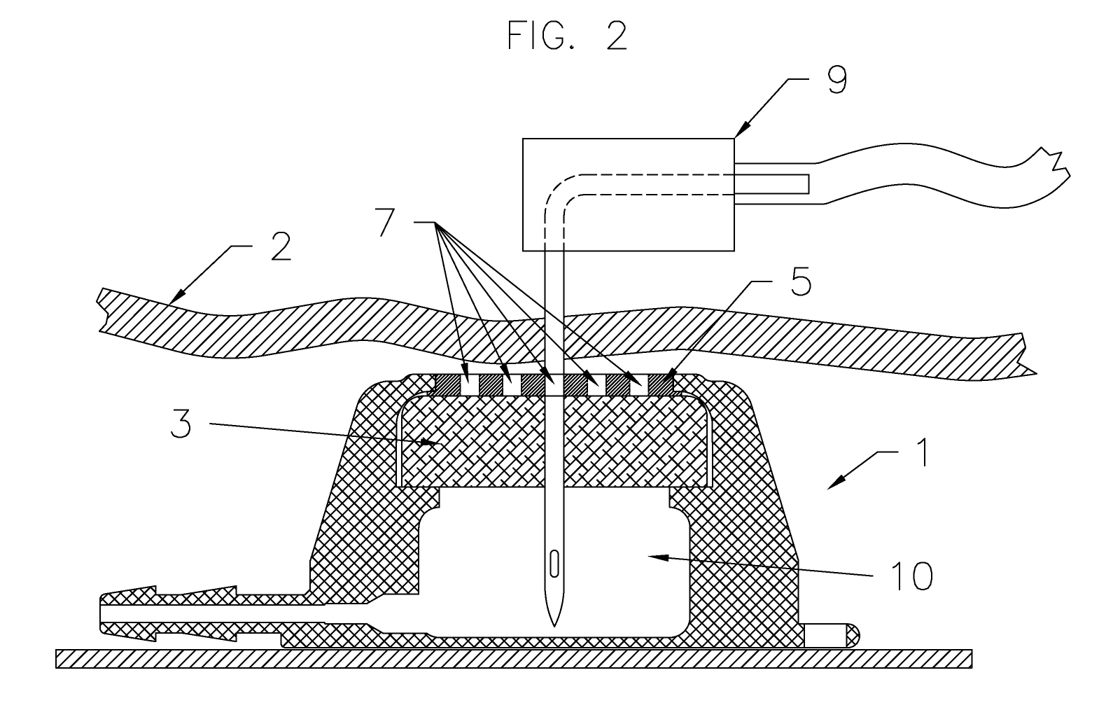

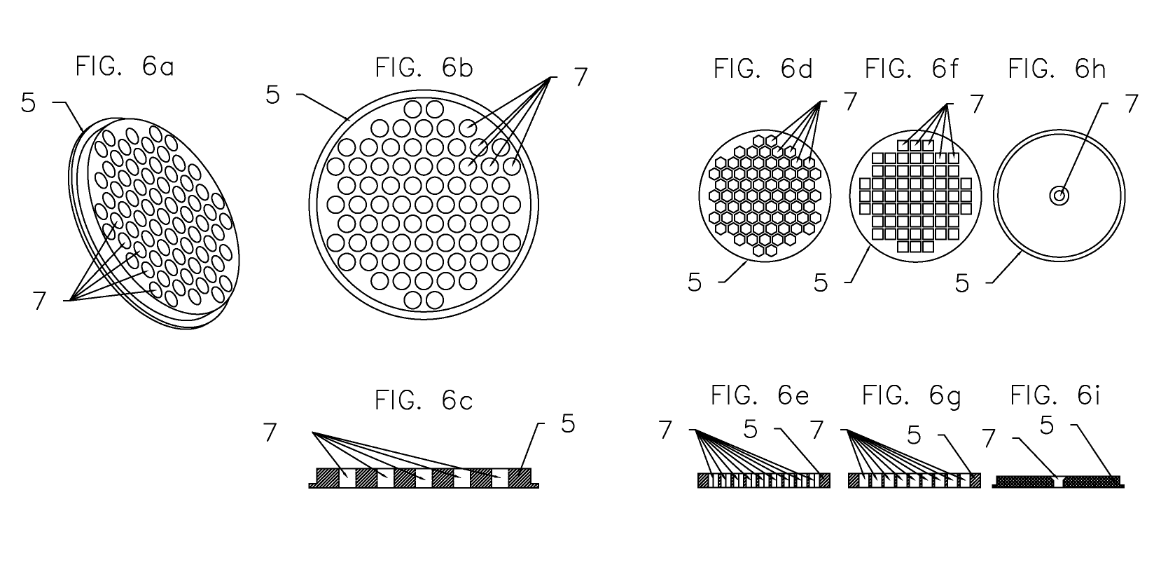

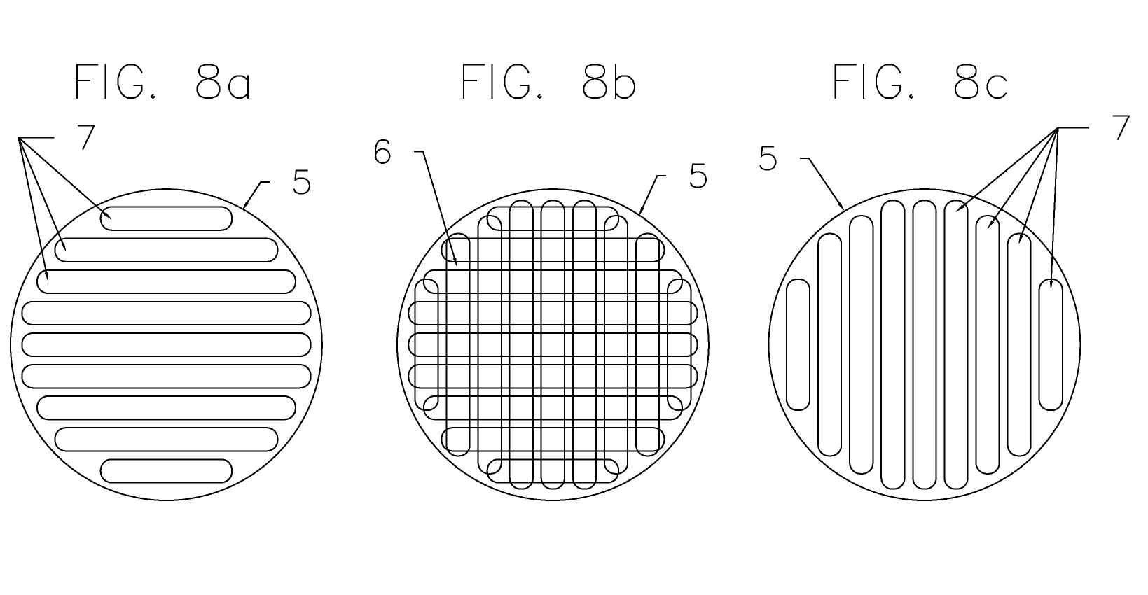

In the present invention, the cover, plate or insert 5, may have a single aperture 7 as shown in FIGS. 1a,b,c and FIGS. 6h,i, or may have a plurality of apertures or holes 7 as illustrated in FIG. 2. The perimeters of the apertures 7 may be circular in shape as shown in FIGS. 6a,b,c; hexagonal in shape as shown in FIGS. 6d,e; rectilinear in shape as shown in FIGS. 6f,g; or may be any other conventional shape or non-conventional shape. The aperture 7 may but is not limited to an opening, through hole, slit or any conventional structure similar to an aperture. In another embodiment, the cover, plate or insert 5 may be slotted as is illustrated in FIGS. 8a,c. In another embodiment of the present invention, the cover, plate or insert 5 may be a wire screen. The aperture 7 may be devoid of material or it may contain material such as polymeric septum material.

The cover, plate or insert 5 may be constructed of a material or combination of materials appropriate for use in an implanted or external medical device. The material may be a metal including but not limited to stainless steel, titanium, Nitinol. Alternatively, the material may be plastic including but not limited to polysulfone, polycarbonate, polyimide, or PEEK. Alternatively, the material may be ceramic.

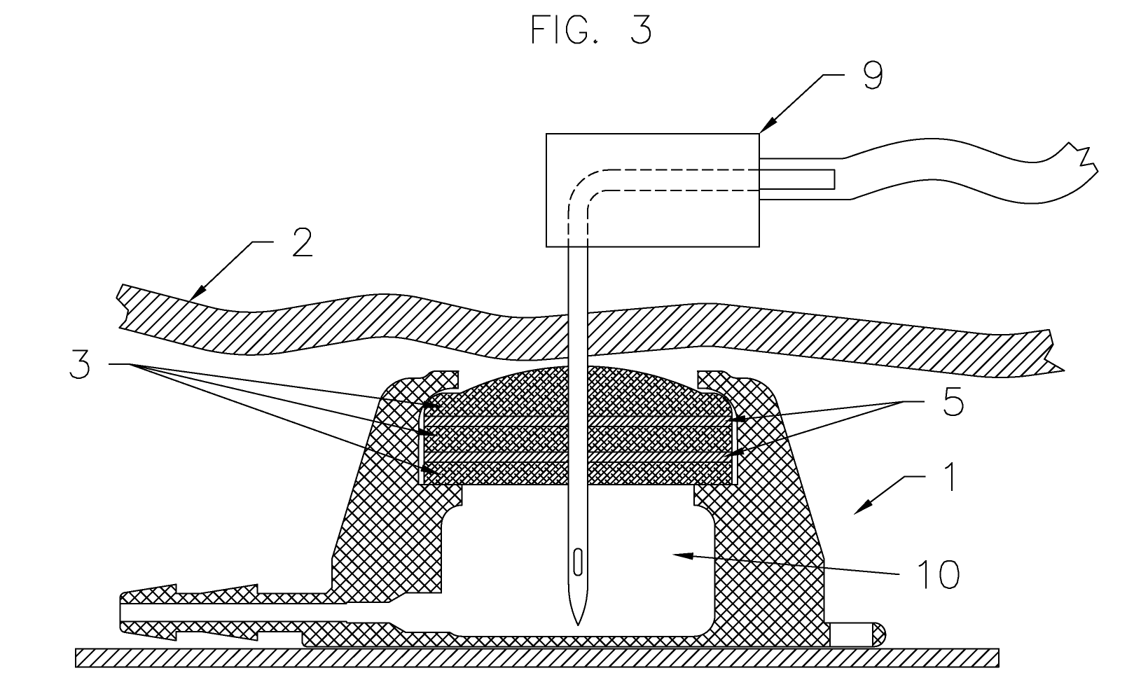

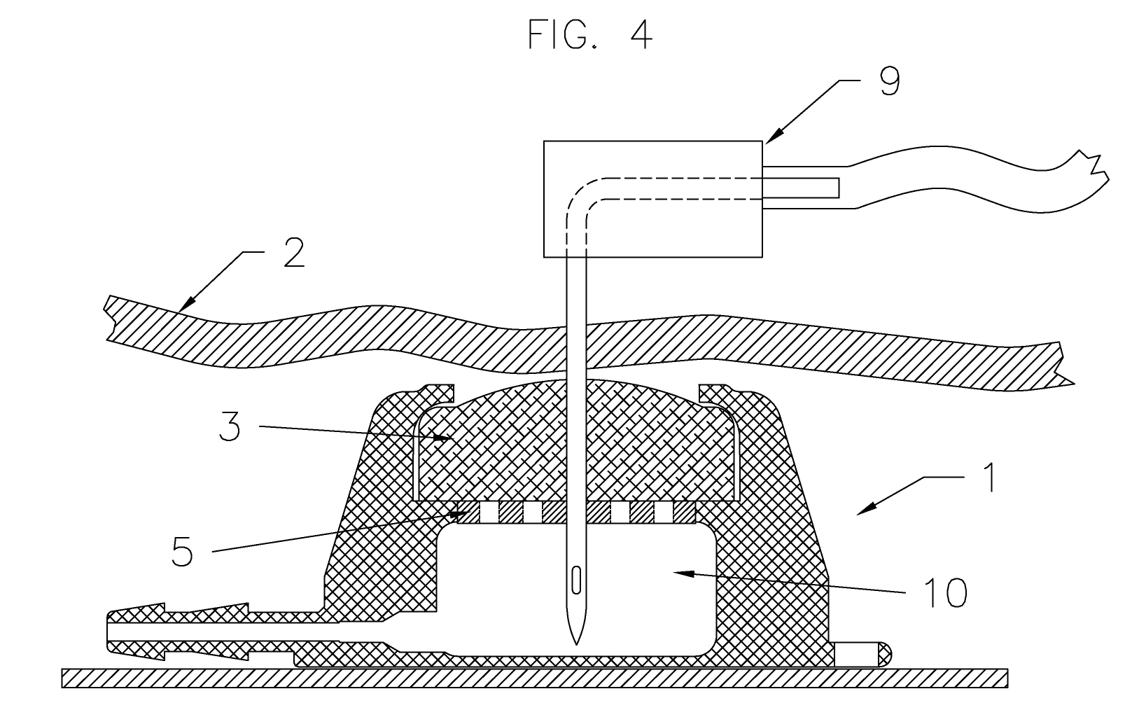

As shown in FIGS. 1a,b,c and 2, the cover, plate or insert 5 may be located on the exterior surface of the septum 3, the surface of the septum 3 located most distal to the reservoir 10. In another embodiment, the cover, plate or insert 5 may be located on the interior surface of the septum 3, the surface of the septum 3 located most proximal to the reservoir 10, as shown in FIG. 4, or within the body of the septum 3 as shown in FIG. 3.

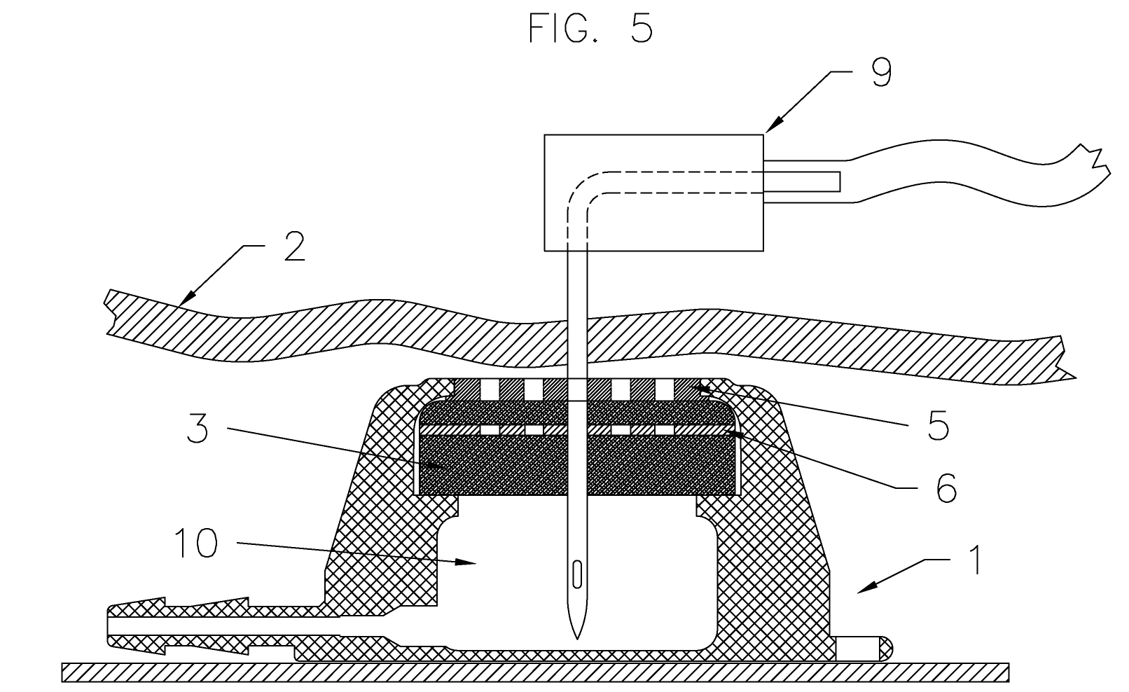

In one embodiment of the present invention, the improved septum-containing device may have a combination of at least two covers, plates or inserts 5. FIG. 3 illustrates an improved septum-containing device having two plates 5 and 6 within the body of the septum 3 at a predetermined distance from each other. FIG. 5 illustrates another embodiment wherein the improved septum-containing device has two plates 5 and 6 wherein one plate is located on the exterior surface of the septum 3 and the other plate is located within the body of the septum 3. In another embodiment, as shown in FIG. 8b, the improved septum-containing device has at least two plates 5 and 6, positioned in parallel planes, having at least one slot 7 wherein one plate has been rotated 90 degrees relative to the other plate.

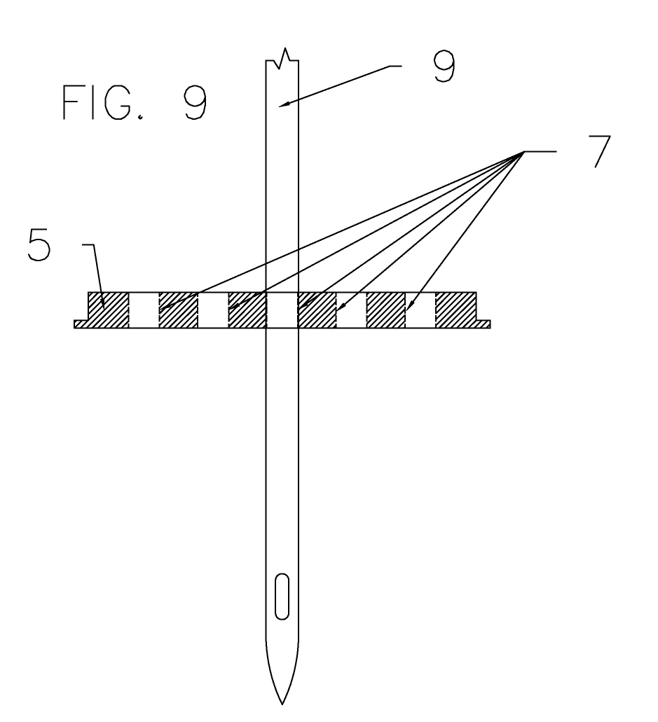

In another embodiment of the invention as shown in FIG. 9, the at least one cover, plate or insert 5 has at least one aperture, opening, slot or through-hole through 7 that is slightly smaller in diameter than the diameter of the corresponding septum-piercing device 9 to allow for an interference fit. Contact force between the perforated plate and the septum-piercing device prevents listing and opposes dislodgement forces.

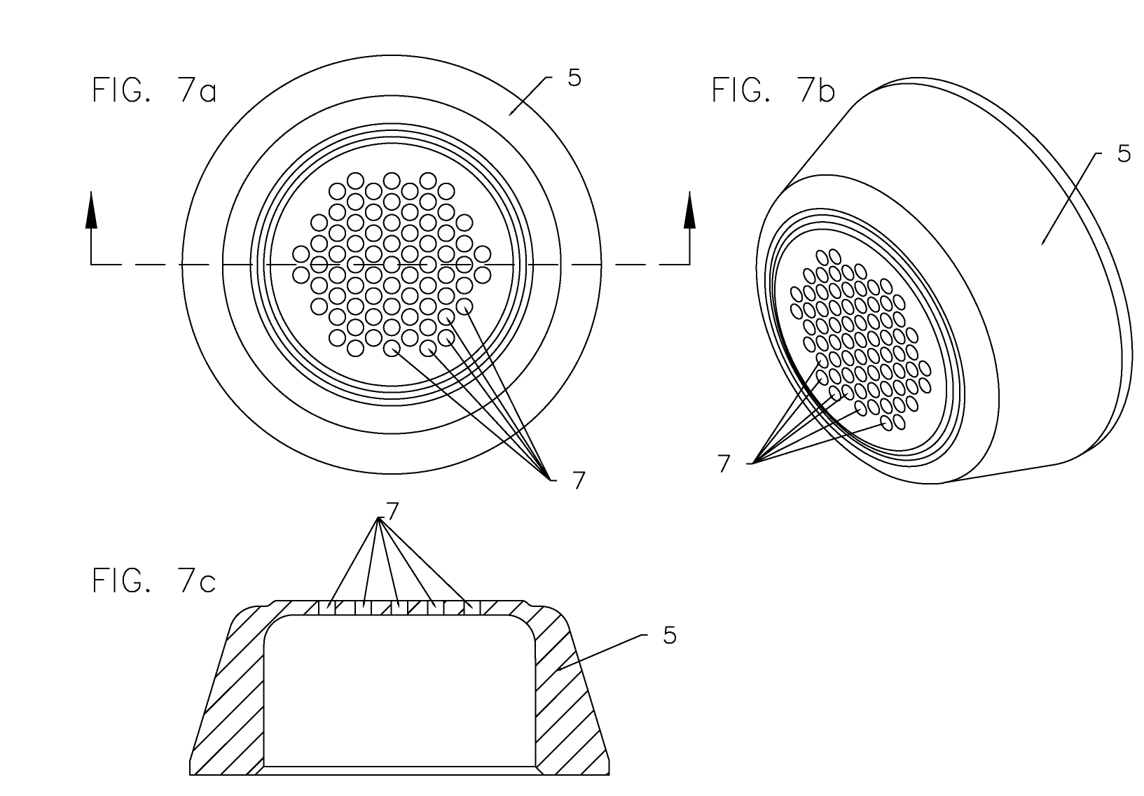

In another embodiment of the present invention, the cover, plate, or insert 5 may be an attachment or component that can be assembled on to a septum-containing device 1, specifically an implanted infusion port, or the cover, plate, or insert 5 may be fabricated as an integral part of a septum-containing device component as shown in FIGS. 7a,b,c. In a preferred embodiment of the present invention, the improved septum-containing device 1, specifically an implanted infusion port, may have a top having a single aperture 7 wherein the top of the improved septum-containing device 1 replaces the cover, plate, or insert 5.

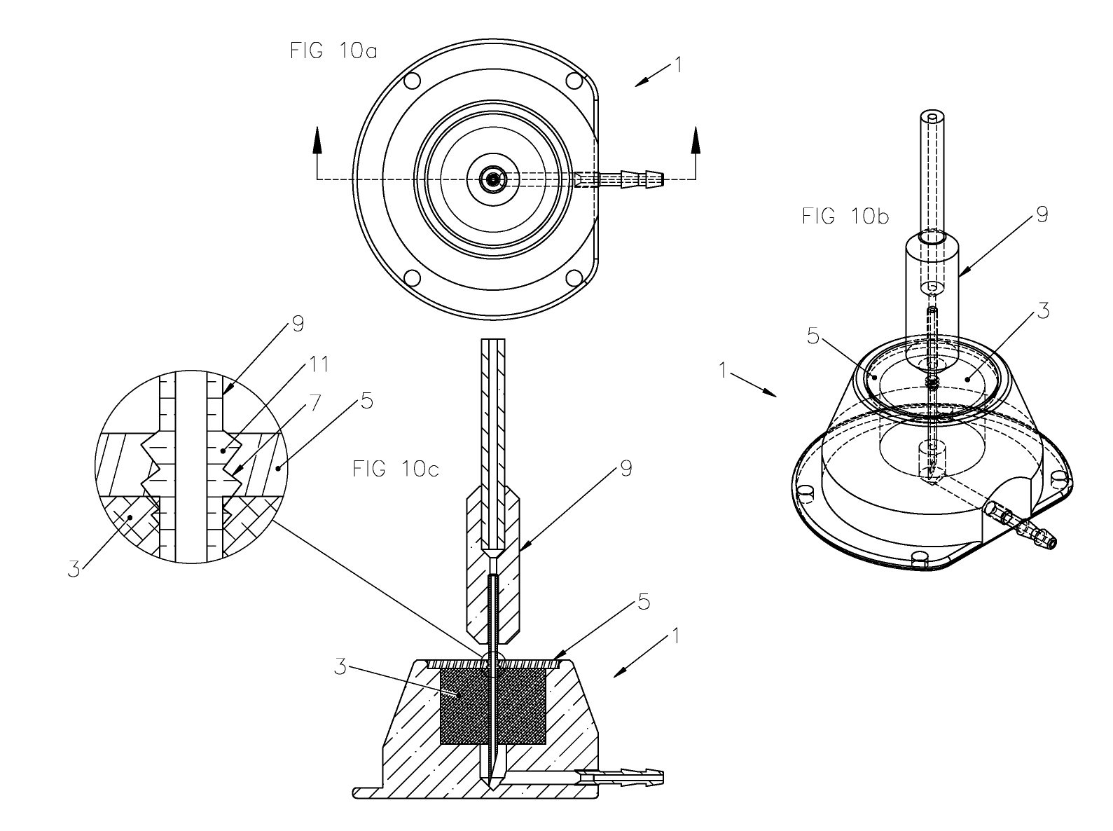

In one embodiment of the present invention, as illustrated in FIGS. 10a,b,c, the at least one cover, plate or insert 5 is intended to lock or secure the septum-piercing device 9 into the improved septum-containing device 1. In this embodiment, the cover, plate or insert 5 may have at least one aperture 7 with a side wall structure that is threaded and matches to a corresponding thread 11 on the exterior surface of the non-coring needle 9 shaft to secure the non-coring needle in the threaded aperture 7. The septum-piercing device 9 or non-coring needle may be further secured into the improved septum-containing device 1 by any conventional locking means, including but not limited to interference fitting between the non-coring needle shaft and at least one aperture 7, ridges or steps on non-coring needle shafts and recessed apertures or holes, and rings or sleeves on non-coring needle shafts to secure or lock the non-coring needle in place.

In another embodiment of the invention, the geometry or design of the septum-piercing device 9 or non-coring needle may have a notch, slot or projection to aid in restraining the non-coring needle from dislodging. Lateral forces result in listing and dislodgement of the non-coring needle. A notch, slot or projection on the non-coring needle, which can be annular or on a single side of the non-coring needle, will engage the at least one aperture, opening, slot or through-hole 7 preventing further dislodgement of the non-coring needle.

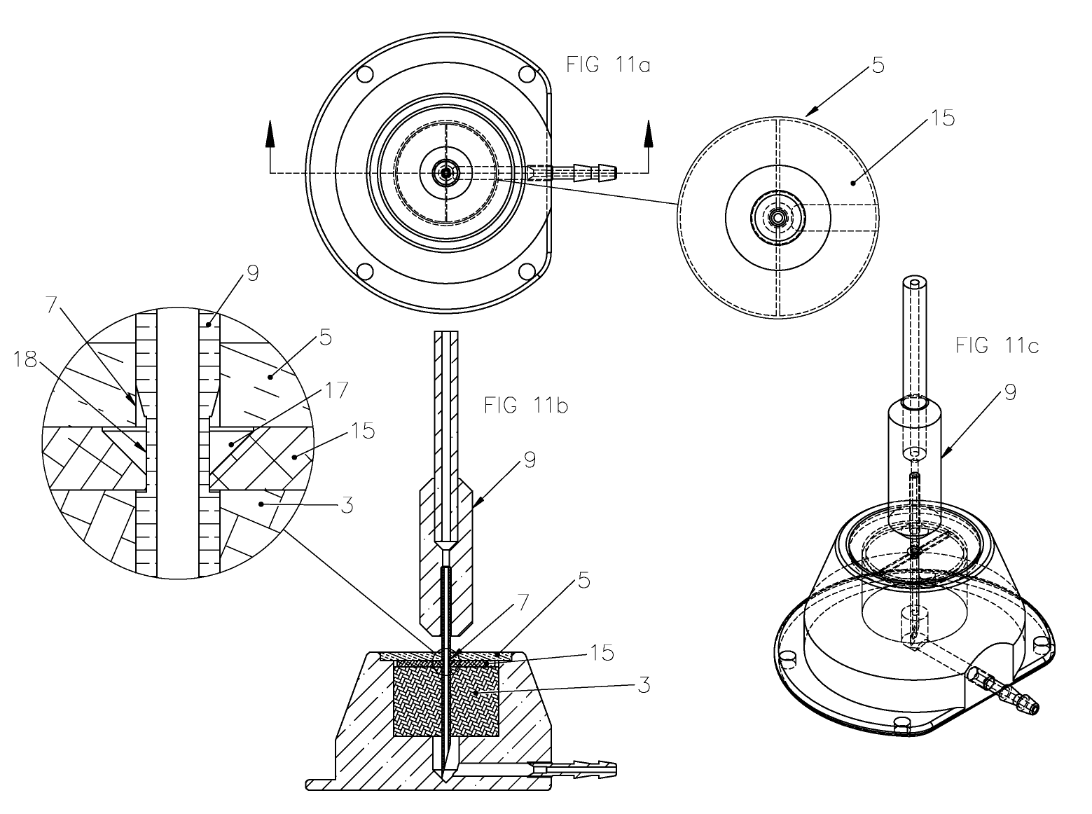

In another embodiment of the present invention, the cover, plate or insert 5 may be split or segmented into at least two segments as shown in FIGS. 11a,c.

FIG. 11 illustrates an embodiment of the present invention wherein the septum-piercing device or non-coring needle has an annulus 18. In a preferred embodiment, the improved septum-containing device 1 may have at least two covers, plates or inserts which include one cover, plate, or insert 5 on the exterior surface of the septum 3 which has a single aperture 7, and a second split or segmented plate 15 which has spring-like properties imparted by the polymeric septum 3, that forms a single aperture 17 when the at least two halves or portions of the split plate 15 spread apart. The split plate 15 is located between the cover, plate, or insert 5 and the septum 3 of the improved septum-containing device 1. When the non-coring needle 9 is inserted through the aperture or aperture 7 of the cover, plate or insert 5, the non-coring needle 9 passes through the aperture 17 formed by the split plate 15 which spreads apart to accommodate the non-coring needle 9 whose outer diameter is larger than the smallest width of the aperture 7 of the split plate 15. The annulus 18 of the non-coring needle is captured between the two halves of the split plate 15. The split plate 15 has spring-like properties that encourage its two halves to return to their resting position thereby gripping the annulus section 18 of the non-coring needle 9 shaft.

In this embodiment, the cover, plate, or insert 5 or split plate 15 may be of any conventional geometry. Further, any conventional springs or spring technology may be incorporated that can apply force to the non-coring needle annulus to reduce the risk of non-coring needle dislodgement.

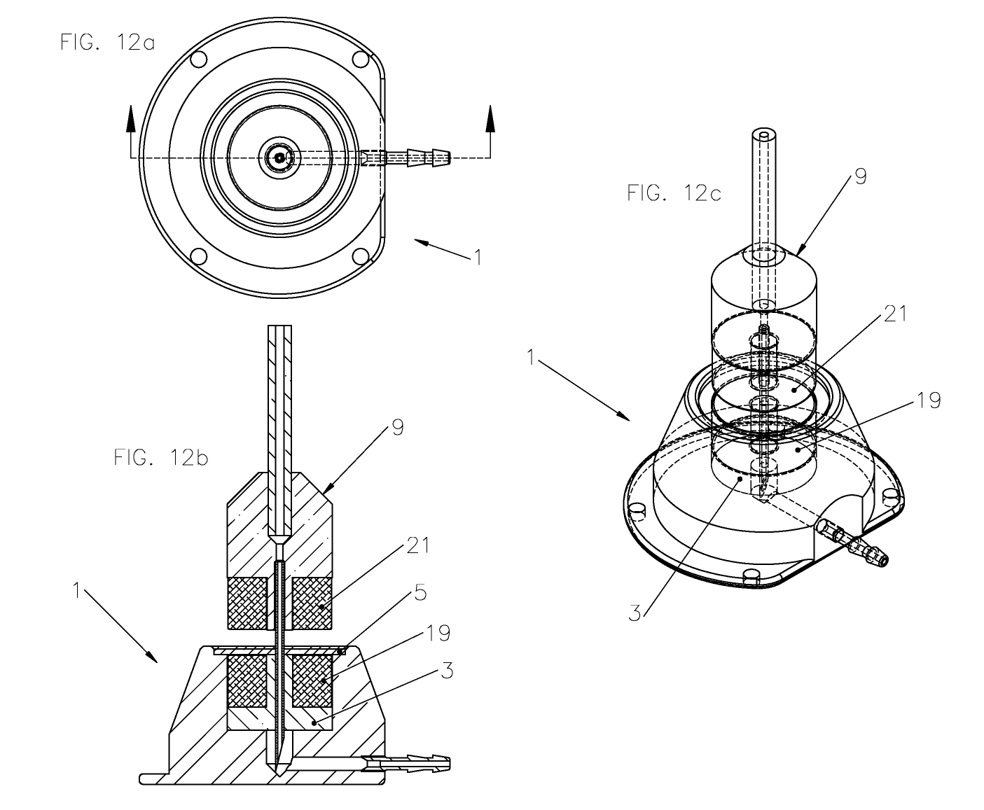

In another embodiment, as shown in FIGS. 12a,b,c, the cover, plate or insert 5 may have a magnet 19 located under the surface of the cover, plate or insert 5 which attracts a magnet 21 attached to the shaft of a non-coring needle in order to create a coupling to lock or secure the non-coring needle in the improved septum-containing device 1. In another embodiment, there may be a cover, plate or insert 5 with a single aperture 7 which guides the non-coring needle to the center of the improved septum-containing device 1 in order to align both the magnet 19 under the surface of the cover plate or insert 5 and the magnet 21 attached to the non-coring needle in such a way as to provide coupling force which in addition to the friction force exerted by the side wall structure of aperture 7 reduces the risk of premature dislodgement.

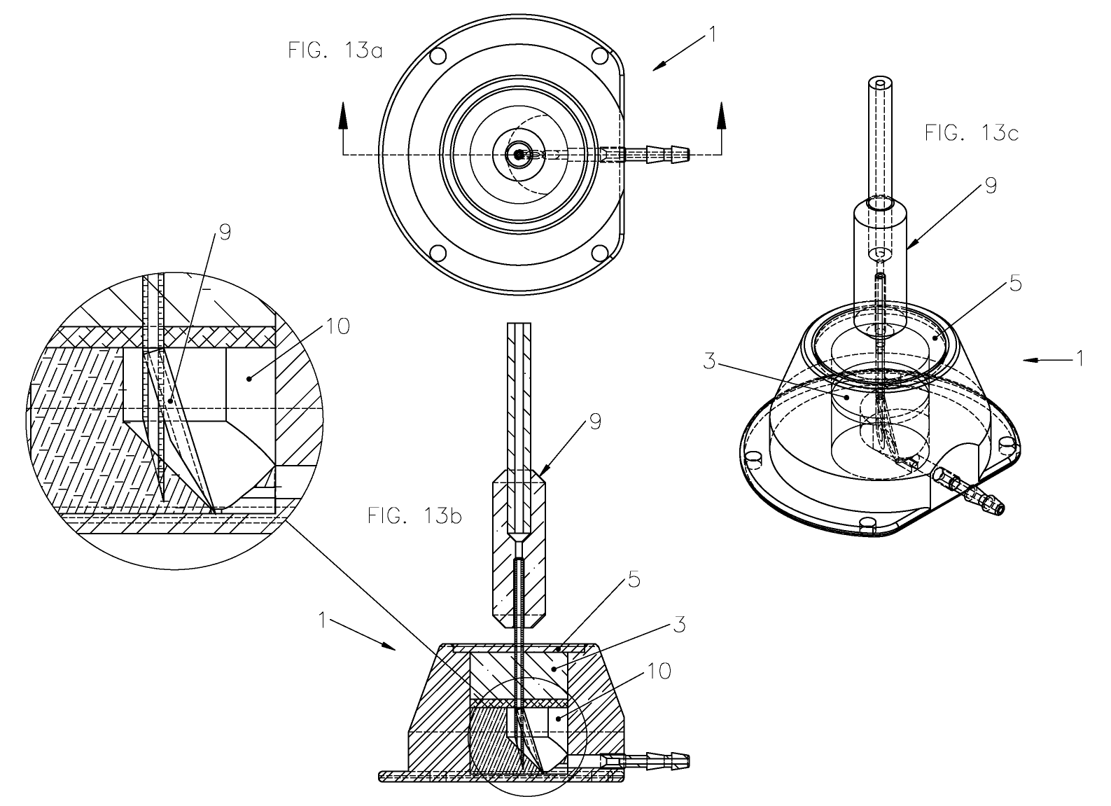

In another embodiment illustrated in FIGS. 13a,b,c, the improved septum-containing device 1 may have a cover, plate or insert 5 located on the exterior surface of the septum 3 and a reservoir 10 with an angled side wall section in the projected path of a non-coring needle 9 which causes the inserted non-coring needle to deflect and deform as it is advancing into the reservoir 10 from its original orientation perpendicular to the cover, plate or insert 5. This deflection and deformation of the non-coring needle 9 increases the force required to remove the non-coring needle 9 from the improved septum-containing device 1 and is thus an effective method for gripping or locking the non-coring needle 9 in the improved septum-containing device 1. In this embodiment, the reservoir may be of any conventional geometry to effect a deflected or deformed needle segment in the reservoir.

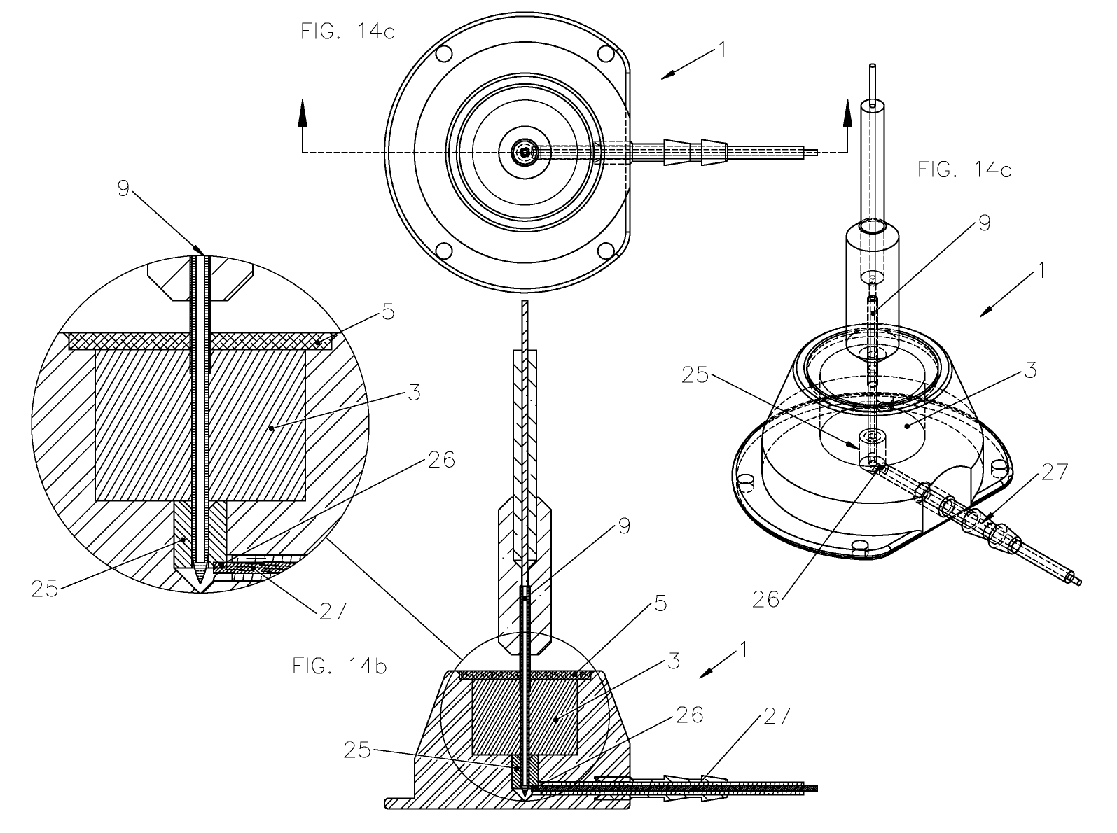

FIGS. 14a,b,c illustrate another embodiment of the present invention wherein the improved septum-containing device 1 has a cover, plate or insert 5 with a single aperture 7 located on the exterior surface of the septum 3, an electrical connector 25 to create an electrical connection between the septum-piercing device 9 and a conductive lead 27 extending from the improved septum-containing device 1 in order to connect to a sensor (not shown), an actuator (not shown), stimulator (not shown), etc. The septum piercing device 9 non-coring needle passes through the aperture 7 to the location of the connector 25 in the improved septum-containing device 1. In another embodiment, the cover, plate or insert 5 may have a plurality of apertures 7 to enable the user to direct multiple septum-piercing devices 9 or non-coring needles to precise locations in the improved septum-containing device 1 to effect multiple electrical connections. Further, the contact between the septum-piercing device 9 and a cover, plate or insert 5 may also effect an electrical connection.

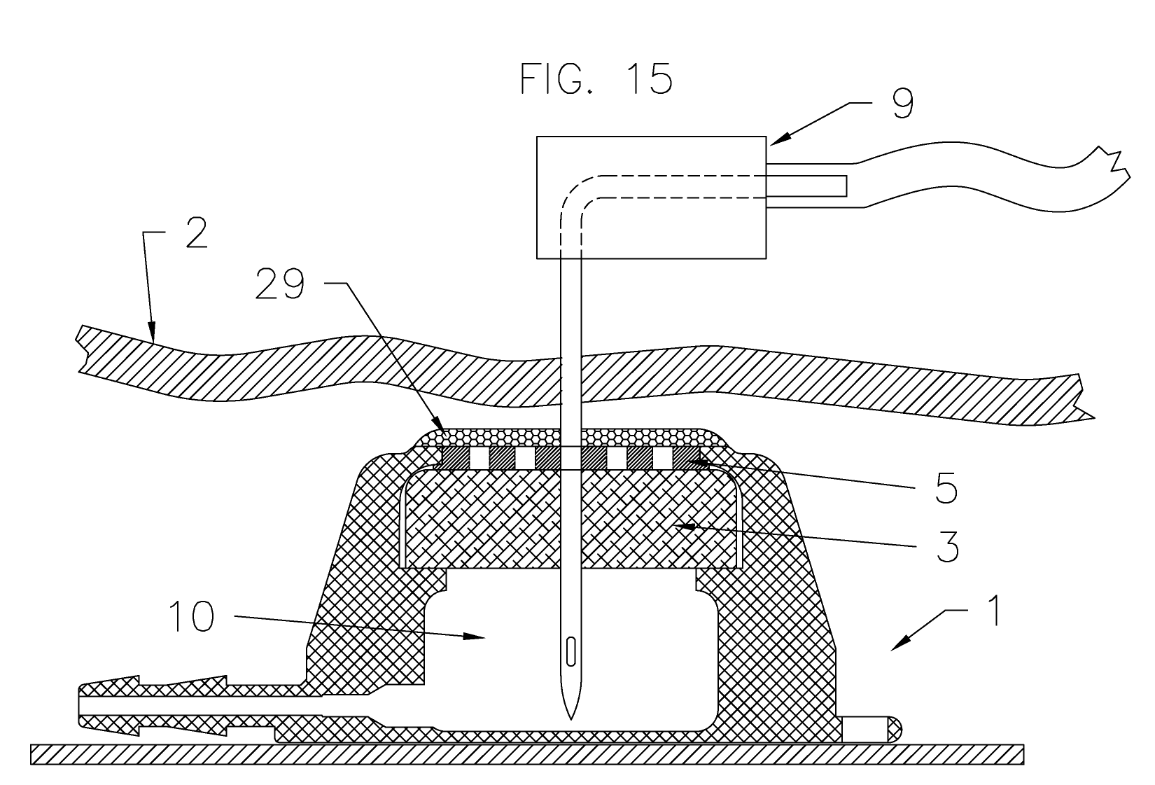

FIG. 15 illustrates another embodiment of the present invention wherein the at least one cover, plate or insert 5 has a penetrable polymeric cover 29 located on the exterior surface of the at least one cover, plate or insert 5 situated on the exterior surface of the septum 3. As the septum-piercing device 9 is inserted into the improved septum-containing device 1, the penetrable cover 29 prevents human or animal skin 2 and tissue from being pushed into and occluding the at least one aperture 7.

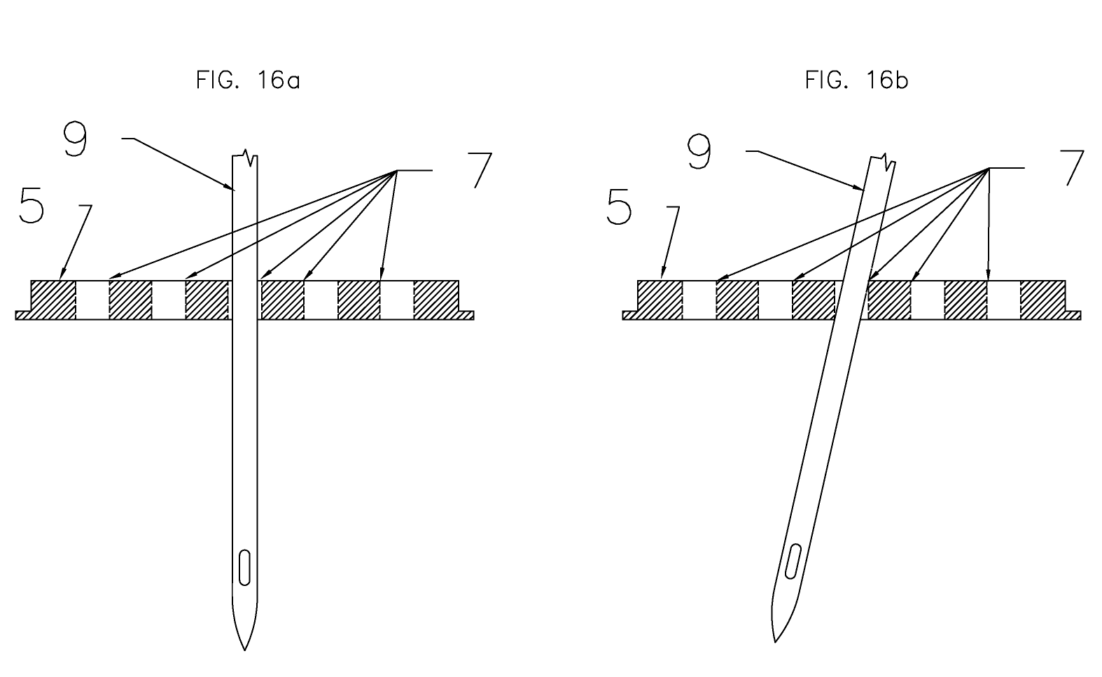

FIG. 16a illustrates the clearance of the septum-piercing device 9 in the aperture 7. A person can orient the septum-piercing device 9 perpendicular to the plate 5. Due to the clearance between the diameter of the septum-piercing device 9 and the aperture 7, the needle passes in and out of the aperture 7 and the improved septum-containing device without difficulty.

FIG. 16b illustrates the contact created between the aperture 7 and the septum-piercing device 9 when the septum-piercing device 9 deviates from a perpendicular orientation to the plate 5. The friction forces created by this contact increase the force required for the septum-piercing device 9 to dislodge from the plate 5 contained in the improved septum-containing device.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. An improved septum-containing medical device comprising:
a septum-containing device having at least one polymeric septum;
at least one plate having at least one aperture;
wherein the side wall of the at least one aperture applies forces to an indwelling septum-piercing device.

2. An improved septum-containing medical device of claim 1, wherein the at least one plate is located on the exterior surface of the polymeric septum.

3. An improved septum-containing medical device of claim 1, wherein the at least one plate is located within the body of the polymeric septum.

4. An improved septum-containing medical device of claim 1, wherein the at least one plate is located on the interior surface of the polymeric septum.

5. An improved septum-containing medical device of claim 1, wherein the at least one plate is has a plurality of apertures.

6. An improved septum-containing medical device of claim 1, wherein the perimeter of the at least one aperture is circular in shape.

7. An improved septum-containing medical device of claim 1, wherein the perimeter of the at least one aperture is rectilinear in shape.

8. An improved septum-containing medical device of claim 1, wherein the perimeter of the at least one aperture is star shaped.

9. An improved septum-containing medical device of claim 1, wherein the at least one plate is slotted.

10. An improved septum-containing medical device of claim 1, wherein the improved septum-containing medical device has at least two plates at a predetermined distance from each other.

11. An improved septum-containing medical device of claim 10, wherein the improved septum-containing medical device has at least two plates in parallel planes and one plate rotated at a predetermined angle relative to each other.

12. An improved septum-containing medical device of claim 1, wherein the improved septum-containing medical device has at least two plates wherein the at least two plates are assembled on top of another with no space separating them.

13. An improved septum-containing medical device of claim 12, wherein the improved septum-containing medical device has at least two plates in the same plane rotated at a predetermined angle relative to each other.

14. An improved-septum-containing medical device of claim 1, wherein the at least one aperture is smaller than the outer diameter of a shaft of a septum-piercing device resulting in an interference fit of the septum-piercing device in the aperture.

15. An improved septum-containing medical device of claim 1, wherein the septum-containing device has a top, and the plate having at least one aperture is integrated into said top.

16. An improved septum-containing medical device of claim 1, wherein the improved septum-containing medical device has at least two plates of which a first top plate has at least one aperture and a second plate which is segmented into at least two pieces form a single aperture.

17. An improved septum-containing medical device of claim 1, wherein the plate has at least one magnet located on the interior surface of the plate.

18. An improved septum-containing medical device of claim 1, wherein the improved septum-containing medical device has at one least plate which is segmented into at least two pieces.

19. An improved septum-containing medical device of claim 1, wherein the septum-containing device has a reservoir having a section of side wall which is in the projected path of the introduction of a septum-piercing device into the septum-containing device.

20. An improved septum-containing medical device of claim 1, wherein the septum-containing device has a reservoir having a bottom wall, side wall and an electrical connector such that when a septum-piercing device is guided to the electrical connector, an electrical connection is created between the septum-piercing device and any other conductive lead extending from the improved septum-containing medical device.

21. An improved septum-containing medical device of claim 1, wherein the at least one plate has at least one penetrable cover located on the exterior surface of the at least one plate.

22. An improved septum-containing medical device of claim 1, wherein the at least one plate has at least one aperture with walls containing threads.

23. An improved septum-containing medical device and system comprising:
a septum-containing device having at least one polymeric septum;
at least one plate having at least one aperture;
wherein the side wall of the at least one aperture has means for locking a septum piercing device into the aperture; and
at least one septum piercing device.

24. An improved septum-containing medical device and system of claim 23, wherein the at least one plate is located on the exterior surface of the polymeric septum.

25. An improved septum-containing medical device and system of claim 23, wherein the at least one plate is located within the body of the polymeric septum.

26. An improved septum-containing medical device and system of claim 23, wherein the at least one plate is located on the interior surface of the polymeric septum.

27. An improved septum-containing medical device and system of claim 23, wherein the at least one plate has a plurality of apertures.

28. An improved septum-containing medical device of claim 23, wherein the improved septum-containing medical device has at least two plates rotated at a predetermined angle relative to each other.

29. An improved septum-containing medical device of claim 23, wherein the improved septum-containing medical device has at least two plates wherein the at least two plates are assembled on top of another.

30. An improved septum-containing medical device and system of claim 23, wherein the septum-containing device has at least one top and the plate is integrated into said top forming at least one aperture.

31. An improved septum-containing medical device of claim 23, wherein the improved septum-containing medical device has at one least plate which is segmented into at least two pieces.

32. An improved septum-containing medical device and system of claim 23, wherein the improved septum-containing medical device has at least two plates of which a first top plate has at least one aperture and a second split plate which is segmented into at least two pieces that form a single aperture.

33. An improved septum-containing medical device and system of claim 23, wherein the septum-piercing device or non-coring needle has an annulus.

34. An improved septum-containing medical device and system of claim 23, wherein the plate has at least one magnet located on the interior surface of the plate.

35. An improved septum-containing medical device and system of claim 23, wherein the septum-containing medical device has a reservoir having a bottom wall, side wall and an electrical connector such that when a septum-piercing device is guided to the electrical connector, an electrical connection is created between the septum-piercing device and any other conductive lead extending from the improved septum-containing medical device.

36. An improved septum-containing medical device and system of claim 23, wherein the at least one plate has at least one protective cover located on the exterior surface of the at least one plate.

37. An improved septum-containing medical device and system of claim 23, wherein the septum-piercing device has a notch on the exterior surface of a shaft of the septum-piercing device.

38. An improved septum-containing medical device and system of claim 23, wherein the septum-piercing device has a projection on the exterior surface of a shaft of the septum-piercing device.

39. An improved septum-containing medical device and system of claim 23, wherein the septum-piercing device has threads on the exterior surface of a shaft of the septum-piercing device.

40. An improved septum-containing medical device and system of claim 23, wherein the septum-piercing device has a magnet on the exterior surface of a shaft of the septum-piercing device.

41. A method of securing a septum piercing device into an improved implanted septum-containing medical device said method comprising:
inserting said septum-containing device below the skin, said improved septum-containing device comprising a septum-containing device having at least one polymeric septum;
at least one plate having at least one aperture wherein the side wall of the at least one aperture applies forces to an indwelling septum-piercing device;
and introducing said septum piercing device through the skin and into said at least one aperture.
